(19) United States
(12) Patent Application Publication
(10) Pub. No.: US 2011/0174222 A1
(43) Pub. Date: Jul. 21, 2011

(54) METHOD FOR REARING SOLDIER FLIES

(76) Inventor: Sang Hoon Lee, Gyeonggi-do (KR)

(21) Appl. No.: 13/002,024

(22) PCT Filed: Jul. 1, 2009
(86) PCT No.: PCT/KR2009/003583

§ 371 Date: Mar. 16, 2011

(30) Foreign Application Priority Data
Jul. 1, 2008 (KR) 10-2008-0063564

(51) Int. Cl. A01K 67/033

(52) U.S. Cl. 119/6.6

(57) ABSTRACT

Provided is a method for producing BSF. The method comprising: providing adult black soldier flies in a breeding farm of black soldier fly; and providing, in the breeding farm, an induction medium comprising sawdust and, on the induction medium, a breeding induction apparatus having a plurality of breeding grooves in which to allow the adult black soldier flies to lay eggs.

### BACKGROUND OF THE INVENTION

1. Field of the Invention

The present invention relates to a method for producing black soldier flies.

2. Description of the Related Art

Organic wastes such as kitchen waste, industrial waste, feces are one of the main causes of serious environmental pollution. These wastes have been recycled as livestock feed or manure, or buried in the landfill, which entails high management cost. However, feeding these wastes livestock without processing generates a concern over epidemics, and composting shows an imbalanced fertilizer effect.

As a solution to the above problems introduced was a waste treatment system which employs insect larvae, such as earthworms, house fly. The cast produced through such treatment system is known to have a high fertilizer effect, thereby improving plant growth and yields.

Especially, for a black soldier fly, the development period from egg to mature adult takes 37˜41 days, and out of those days the larvae, being a decomposer, spends 14 days, during which the larvae decomposes organic wastes. After 14 days, the black soldier fly larvae migrates to an arid place in order to pupate.

In an experiment, 5000 number of the black soldier fly larvae inputted into a 10 kg of organic wastes decompose more than 80% of the organic wastes within 5 days, thereby reducing volume by 42% and weight by 70% of the wastes. It is more efficient to rear the black soldier fly than earthworm and house fly since the black soldier fly larvae spend longer period in decomposing organic wastes. Accordingly a massive producing system for the black soldier fly is demanded.

### SUMMARY OF THE INVENTION

The present invention aims to provide apparatus and method for massive culturing of the black soldier fly.

Additional objects of the present will also be driven without difficulty through the following description.

One aspect of the present invention is a producing method for black soldier flies, the method comprising: providing adult black soldier flies in a breeding farm of black soldier fly; and providing, in the breeding farm, an induction medium comprising sawdust and, on the induction medium, a breeding induction apparatus having a plurality of breeding grooves in which to allow the adult black soldier flies to lay eggs.

In one example embodiment, the breeding farm may comprise a breeding net with 80˜100 meshes to prevent invasions from the outside.

In one example embodiment, the breeding induction apparatus may be formed of flower foam or wood.

In one example embodiment, the breeding grooves may be formed on either or both sides of the breeding induction apparatus.

In one example embodiment, open side of the breeding groove may have a geometric shape selected from the group consisting of round shape, elliptical shape, and polygonal shape.

In one example embodiment, a diameter of the open side of the breeding groove may range from 3 mm to 5 mm.

In one example embodiment, a depth of the breeding groove may range from 7 mm to 10 mm.

In one example embodiment, a providing density of adult black soldier flies may range from 8,000 to 12,000 (cm³/head).

In one example embodiment, the providing a breeding induction apparatus may be performed 3 days after performing the providing adult black soldier flies.

In one example embodiment, the induction medium may comprise a combination of organic waste and sawdust or rice husk in the ratio of 3:1.

In one example embodiment, the induction medium may comprise a combination of organic waste and calf starter and sawdust in the ratio of 2:1:1.

In one example embodiment, the breeding farm may further comprise a stench removing unit to remove a stench emitting from the inside.

In one example embodiment, the method may further comprise rearing pupae of black soldier flies in a rearing medium comprising sawdust of which a relative humidity ranges from 0% to 40%.

In one example embodiment, the rearing may be performed under a condition that a density of pupae ranges from 5,000 to 10,000 (head/240 cm²), temperature is 27±3° C., and the rearing medium comprises a combination of organic waste and sawdust in the ratio of 3:1.

In one example embodiment, the organic waste may be crushed to be combined.

In one example embodiment, the method may further comprise, posterior to the supplying adult black soldier flies, spraying water in the breeding farm two or three times a day to supply water.

In one example embodiment, the breeding farm may be situated indoors and further comprises an air conditioner to maintain temperature of the breeding farm at 29±5° C.

In one example embodiment, the method may further comprise removing a stench emitting from the rearing medium by a stench removing unit.

In one example embodiment, the breeding farm or the rearing medium may be situated in a vinyl greenhouse having an illuminator or in a building with a transparent ceiling to maintain a photoperiod of the rearing medium at 14L:10D.

In one example embodiment, the number of induction medium in a breeding farm of black soldier fly is 8 and temperature of the rearing medium is maintained at 27±3° C. by an air conditioner.

### BRIEF DESCRIPTION OF THE DRAWINGS

The objects, features and advantages of the present invention will be more apparent from the following detailed description in conjunction with the accompanying drawings, in which:

FIG. 1 is a perspective view of a breeding induction apparatus which is used to produce black soldier flies according to an embodiment of the present invention.

FIG. 2 is a plan view of a breeding induction apparatus which is used to produce black soldier flies according to an embodiment of the present invention.

FIG. 3 is a front view of a breeding induction apparatus which is used to produce black soldier flies according to an embodiment of the present invention.

FIG. 4 is a groove generating apparatus by which to generate grooves of a breeding induction apparatus which is used to produce black soldier flies according to an embodiment of the present invention.

FIG. 5 is a flow chart of a black soldier fly producing method according to an embodiment of the present invention.

### DETAILED DESCRIPTION OF EXEMPLARY EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein. Accordingly, example embodiments are to cover all modifications, equivalents, and alternatives falling within the scope of the invention.

It will be understood that, although the terms first, second, etc. may be used herein to describe various elements, these elements should not be limited by these terms. These terms are only used to distinguish one element from another.

It will be understood that, when a feature or element is referred to as being “connected” or “coupled” to another feature or element, it can be directly connected or coupled to the other element or intervening elements may be present. In contrast, when a feature or element is referred to as being “directly connected” or “directly coupled” to another element, there are no intervening elements present.

The terminology used herein is for the purpose of describing particular embodiments only and is not intended to be limiting of example embodiments of the invention. It will be understood that the terms “comprises,” or “includes,” when used herein, specify the presence of stated features, integers, steps, operations, elements, and/or components, but do not preclude the presence or addition of one or more other features, integers, steps, operations, elements, components, and/or groups thereof.

Like numbers are used throughout the drawings to refer to the same or like parts and a repetitive explanation will be omitted. Detailed descriptions of well-known functions and structures incorporated herein may be omitted to avoid obscuring the subject matter of the present invention.

Also, to illustrate the configuration of the apparatus and the steps of the method, will be referred experimental conditions under which an experiment was performed while the invention was in a conceptual stage

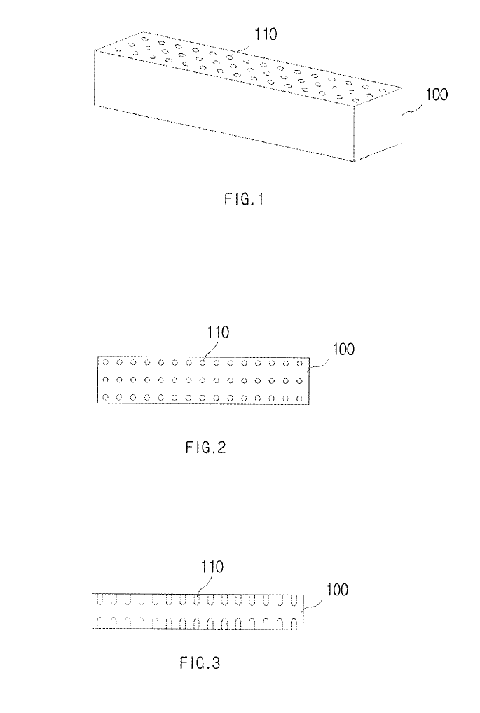

FIGS. 1 through 3 are perspective, plan and front views of a breeding induction apparatus which is used to produce black soldier flies according to an embodiment of the present invention. For convenience, the following illustration will be focused on a case where the organic wastes are food wastes.

The breeding induction apparatus 100 may be shaped of a rectangular parallelepiped elongated in a direction. A breeding farm where the breeding induction apparatus 100 is disposed may comprise a blocking net with 80 to 100 meshes in order to block invasions from the outside. The breeding induction apparatus 100 may be formed of flower-foam or wood.

Also, on at least one side of the breeding induction apparatus 100 are formed breeding grooves 110 with a predetermined depth. An open side of the breeding groove 110 may have a geometric shape selected from the group consisting of round shapes, elliptical shapes, polygonal shapes (e.g. triangle, quadrangle). A diameter of the open side of the breeding groove 110 may range from 3 mm to 5 mm, and a depth of the breeding groove 110 may range from 7 mm to 10 mm.

The breeding groove 110 may be formed by a groove forming apparatus shown in FIG. 4. The groove forming apparatus comprises a main body unit 410, a projection unit 415, and a handle 420. A user may generate the breeding groove 110 by pressing the projection unit 415 against the breeding induction apparatus 100.

A density of adult black soldier flies inputted in the breeding farm may range from 8000 cm³/head to 12,000 cm³/head. The breeding induction apparatus 100 may be disposed 3 days after the adult black soldier flies are inputted.

Here, the breeding induction apparatus 100 may be placed on an induction medium which comprises a combination of food waste and sawdust or rice husks in the proportion of 3:1.

Black soldier fly pupae may be reared in the induction medium with a 0˜40% relative humidity under a physical condition that temperature is 27±3° C., and pupae density is 5,000˜10,000 head/240 cm².

The breeding induction apparatus 100 may be formed such that a height is 50 mm, a width is 40 mm, and a space between the breeding grooves 110 is 15 mm.

A total of 8 breeding induction apparatuses 100 may be disposed in the breeding area.

Here, a size of the breeding area may be 4000*2000*2000 mm (W*D*H).

The black soldier fly larvae can digest food waste for approximately 15 days to the maximum. It takes 3˜5 days for the larvae to digest approximately 80% of food waste provided, thereby reducing the volume by 42%, and the weight by 70%. A digest capacity of a lava is 2˜3 g, and an adult black soldier fly lays a total of 1,000 eggs.

The black soldier fly mainly inhabits a storage area of organic wastes such as food waste storage, a compost storage around livestock barn, etc. Black soldier flies may be collected directly or by using an induction trap. The induction trap may employ rice bran, oil cake, calf starter, etc.

A life cycle of the black soldier fly under the temperature of 27° C. consists of egg stage (4˜5 days), larval stage (14 days), pupal stage (14 days), and adult stage (5˜8 days), so that the lifespan is approximately 37˜41 days. It takes 3˜5 days for a lava to digest food wastes.

Looking at a change in the weight and volume of the food wastes according to time lapse, the weight was reduced by 80% in two days after black soldier larvae were inputted, and by 70% in five days. The volume was reduced by 60% in two days, and by 40% in five days.

In an embodiment, the blocking net was formed to have a size of 1.2 m×1.2 m×1.2 m (W×D×H) (size 1), and a size of 4.0 m×2.0 m×2.0 m (size 2). A density of the blocking net was 200 mesh/inch, which is capable of preventing small insects from invading. It is recommended that the blocking net is larger than size 2, and the blocking net smaller than size 1 was not effective. Sugared water (5% of concentration) was provided on the ground to supply adult flies with water.

The breeding induction apparatus 100 is formed of flower foam or wood on which grooves are generated. The breeding box in which an induction medium is filled has a size of 60 Cm×40 Cm×15 Cm (W×D×H). An average breeding rate is 737±243 eggs/adult. A main ingredient of the medium is food wastes, and the medium should be boiled to be sterilized.

Looking at the breeding aspect (a five hundred of adults observed (under a condition that a proportion of female to male is 2:3), temperature kept at 29±5° C.), adult black soldier flies started to breed in 7 days after developing into an adult, reaching a peak in 9˜10 days. It took 5.2±1.5 days for an egg to hatch, and the hatch rate was 100±0.1%.

An average lifespan of an adult black soldier fly was 15.1±6.0 days (a five hundred of adults (under a condition that a proportion of female to male is 2:3) observed, temperature being kept to 29±5° C.).

Also, black soldier fly larvae were separately reared under two different rearing mediums. In a case (A), the rearing medium was composed of food wastes only, but in a case (B), the rearing medium was composed of food wastes and sawdust in the proportion of 4:1. (In both cases, a rearing temperature and a light condition were 27±3° C., and 14L:8D, respectively).

Qualities of larvae reared in two different conditions are compared in table 1. Here, during a pupating season, escaping pupae can be captured by a covering net.

| | Characters of larvae | | |
|---|---|---|---|
| Classification | Height (mm) | Weight (g/100 head) | Period (day) |
| medium (A) | 20.2 ± 0.8 | 19.5 ± 0.5 | 25~40 |
| medium (B) | 20.4 ± 0.8 | 19.3 ± 0.3 | 20~40 |

[ [symbol] 1] Comparison in larvae characters

An outdoor rearing farm has a size of 4 m×2 m, a protecting net is needed to protect from bird attacks.

Pupae of the black soldier fly may be stored in a box which is formed of a material (e.g. plastic box). As an example, the pupae may be stored in a transparent box filled with sawdust (approximately 70% humidity).

Imagoes are put onto pasture in the breeding farm, a pupa develops into an adult in from 15 to 20 days after pupation, an emergence rate is 88.4±3.2% (repeated three times with a five hundred of pupae, temperature being kept at 27±3° C.).

Imported in the early 1990's, black soldier flies were spread nationwide, occurring on organic wastes such as outdoor bathrooms, manure, food wastes, compost, etc. Their larvae feed on decaying organic matter including animal corpses, manure, plant remains, kitchen wastes, etc.

The black solider fly does not bite livestock or human, and not transmit diseases.

Therefore, the black soldier fly has not been reported as a pest because it has a short lifespan and the adult is not attracted to human habitation or foods.

Also, Female house flies avoid ovipositing where the black soldier flies inhabit, so that populations of house flies can be controlled.

In this embodiment of the present invention, a larger space than 3 m×2 m×2 m (W×D×H) is required for a breeding farm. Density for a net which keeps the black soldier flies from natural enemies is within 80 meshes.

In order to make better use of a space, the breeding induction apparatus is built to have a two-tier structure. Although adults do not need to feed, water is sprayed 2˜3 times a day for water supply.

The breeding induction apparatus is disposed on an induction medium (food waste:fermented calf starter:sawdust=2:1:1). The apparatus is formed of flower foam or wood, on either or both sides of which the above described grooves are generated to induce breeding. This is based on the idea that the adult black soldier fly lays eggs in hidden crevices. Four or five apparatuses (per 240 cm²) are disposed.

The apparatus made of wood showed a higher breeding rate. The flower foam is reusable 4˜5 times, but the wood can be used semipermanently.

Looking at the breeding rate according to the size of the groove, when a diameter of an open side of the groove was 5 mm, 4 mm, and 3 mm, the breeding rate was 60%, 36.8%, and 31.7%, respectively.

In the case where the open side has elliptical shape or polygonal shape, the diameter may be a diameter of either incircle or circumcircle of the ellipse or the polygon. Further, in the case of the elliptical shape, the diameter may be determined by multiplying 2 by an average value of the major and minor axes.

According to this embodiment, the breeding grooves with 3 mm-5 mm diameter showed a highest breeding result.

Looking at a breeding rate according to a depth of the breeding groove, when a depth of the breeding groove was 6 mm, 7 mm, 8 mm, 9 mm, 10 mm, and 11 mm, the breeding rate was 8%, 23%, 18%, 17%, 22%, and 4%, respectively, showing a highest result when the depth was 7˜10 mm.

Looking at breeding amount according to the type of the induction medium, the number of egg masses was 300 in the case of food waste, 330 in the case of calf starter, 190 in the case of oil cake, and 50 in the case of rice husks, suggesting that food waste and fermented calf starter were most effective. In addition, rotten food waste was preferred to fresh waste.

Looking at the breeding amount according to the amount of induction mediums inputted, one column of rearing medium per 1 m² is preferable when the size of the breeding farm is 4 m×2 m×3 m (D×W×H) and the number of adults inputted is 2,000.

Looking at the breeding amount according to the number of adults inputted in the breeding farm, 2,000˜3,000 flies are preferable when the size of the breeding farm is 4 m×2 m×3 m (D×W×H) and the number of columns of the rearing medium is 8. In other words, a preferable input density of the adult flies is 8,000˜12,000 cm³″head. The number of egg masses was 276, 714, and 842, when there were 1,000, 2,000, and 3,000 adult flies, respectively.

A preferable rearing density for artificially multiplying black soldier fly is suggested as in the table 2.

TABLE 2
Preferable rearing density for BSF

| | | |
|---|---|---|
| REARING DENSITY | HEIGHT (mm) | PUPATION |

| (HEAD/240 cm²) | LAVA | PUPA | RATE (%) |
|---|---|---|---|
| 5,000 | 20.7 ± 1.1 | 19.2 ± 1.1 | 86.9 |
| 10,000 | 18.2 ± 1.6 | 18.2 ± 1.3 | 76.4 |
| 15,000 | 17.3 ± 1.4 | 16.6 ± 1.4 | 81.1 |
| 20,000 | 17.8 ± 1.5 | 17.1 ± 1.4 | 53.2 |

A rearing box was sized of 60 cm×40 cm×15 cm, and a rearing condition was 27° C., R.H. 60%. The qualities of larvae and pupae were superior in the groups where the density was 5,000 and 10,000, and a pupation rate was highest in the group where the density was 5,000. Therefore, it is preferable that the rearing density is less than 10,000 heads/cm².

An emergence rate according to a protection condition is as follows:

While keeping a protection temperature at 27° C., the emergence rate was observed (1) when no medium (sawdust) was added, (2) a medium with a 0% of humidity was added, (3) a medium with a 20% of humidity was added, and (4) a medium with a 40% of humidity was added.

The emergence rate was 44.9%, 92.5%, 93.4%, and 94.7%, in the cases of (1), (2), (3), and (4), respectively. This result suggests that a medium is necessary and the humidity is recommended to be 20% approximately.

According to an embodiment of the present invention, pupae of the black soldier fly may be stored at a low temperature. When the pupae were stored in a dark condition at a temperature of 10° C., an emergence rate were 98% initially, 98.8% after 10 days, 86.6% after 20 days, and 41.7% after 30 days.

Also, a digestion capacity for food waste of the black soldier fly was calculated.

TABLE 3
DIGESTION CAPACITY OF BSF

| | THE | | |
|---|---|---|---|
| | NUMBER | | DIGESTION |
| GROUP | OF | TOTAL AMOUNT | CAPACITY/ |
| CLASSIFICATION | LARVAE | OF DIGESTION (g) | HEAD (g) |
| GROUP 1 | 879 | 2000 | 2.30 |
| GROUP 2 | 938 | 3000 | 3.20 |
| GROUP 3 | 785 | 2000 | 2.55 |
| GROUP 4 | 893 | 1400 | 1.68 |
| GROUP 5 | 1389 | 1000 | 1.08 |
| Mean ± S.D. | 977 ± 237 | 1880 ± 756 | 2.2 ± 0.8 |

Groups 1 through 5 are distinguished by the number of inputted larvae.

As shown in Table 3, 2˜3 g of food waste is digested by one lava, which may be converted into 2˜3 kg per (female) adult (one female imago lays approximately 1,000 eggs).

According to an embodiment, an indoor BSF rearing apparatus may be formed of plastic box, and sized of 60×40×15 cm. A rearing condition may be 27° C., R.H. 60%, and a rearing density may be 5,000˜10,000 head/box.

When rearing larvae, the rearing box may be covered by a net, which prevents attacks from natural enemies. Because grown up lavae (prepupae) escape from the rearing box, pupae need to be collected. The collected pupae are inputted in a protective apparatus to emerge (Condition: 27° C., R.H. 60%, photoperiod of 14L:10D).

A preferable condition for a rearing medium for mass rearing may be as follows:

Firstly, food waste is crushed to enhance digestion efficiency. Additional agent (saw dust or rice husk) is mixed to the crushed food waste in the ratio of 1:3. Further, 2˜3 kg of Food waste may be supplied daily or every other day.

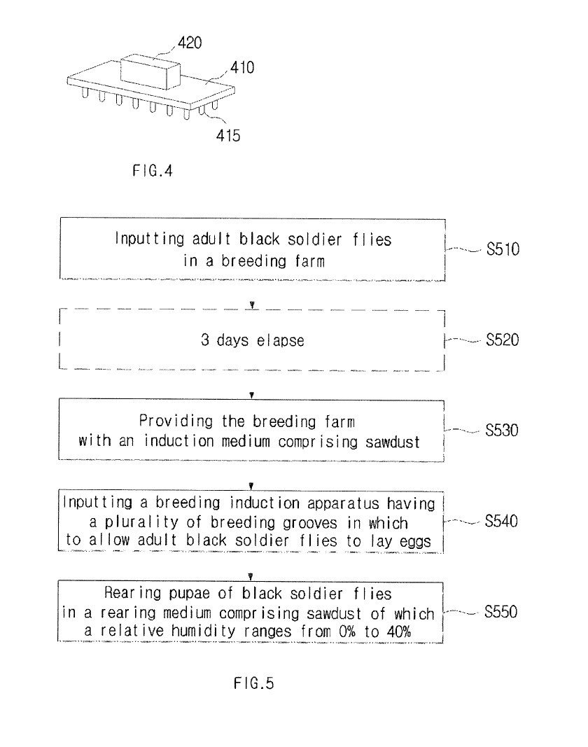

FIG. 5 illustrates a flowchart of a BSF producing method according to an embodiment of the present invention.

At step S510, adult black soldier flies are inputted in a breeding farm. An input density may range from 8,000 to 2,000 (cm³/head)

At step S520, three days elapse before an induction medium is provided in the breeding farm at step S530. The induction medium may comprise a combination of food waste and sawdust or rice husks in the ration of 3:1.

At step S540, on the induction medium is disposed a breeding induction apparatus where a plurality of breeding grooves are formed to allow the adult black soldier flies to lay eggs in the grooves. Here, the induction medium and the breeding induction apparatus may be disposed at the same time.

At step S550, in order to allow pupae to emerge, pupae of the black soldier flies are reared in a rearing medium comprising sawdust of which relative humidity ranges from 0% to 40%. Here, the rearing medium may comprise a combination of food waste and sawdust in the ratio of 3:1. Temperature may be maintained at 27±3° C., and density of pupae may range from 5,000 to 10,000 (head/240 cm²).

Further, in order to satisfy the above temperature condition (for pupation or emergence, 27±3° C., and for examination of the number of egg masses according to time lapse, 29±5° C.), the breeding farm or the rearing medium may be disposed indoors (e.g. a vinyl greenhouse).

Also, in order to maintain the above temperature condition, the breeding farm or the rearing medium may further comprise an air conditioner, and the air conditioner may comprise a cooling unit and a heater to control the temperature. Also, the breeding farm may further comprise an illuminator to satisfy the above photo period (for rearing black soldier fly larvae, 14L: 10D).

Further, in this embodiment, a stench removing unit may be added to remove stench emitting from the breeding farm or the rearing medium. Inhaled gas may be discharged to the outside.

For instance, the stench removing unit may comprise an inhalant pan to inhale stench, and a deodorizer such as deodorizing microorganism, ozone, activated carbon, naphthalene, charcoal, etc. to remove the inhaled stench. Also, the stench removing unit may employ a catalyst cell comprising a special uvx lamp and an alloyed metal which is a combination of rhodium, silver, copper, and titanium dioxide, thereby generating purifying ions such as OH radicals, superoxide anion, etc. to remove harmful bacteria and virus.

Although exemplary embodiments of the present invention have been described in detail hereinabove, it should be clearly understood that many variations and modifications of the basic inventive concepts herein taught which may appear to those skilled in the present art will still fall within the spirit and scope of the present invention, as defined in the appended claims.

1. A producing method for black soldier flies, the method comprising:
providing adult black soldier flies in a breeding farm of black soldier fly; and
providing, in the breeding farm, an induction medium comprising sawdust and, on the induction medium, a breeding induction apparatus having a plurality of breeding grooves in which to allow the adult black soldier flies to lay eggs.

2. The method of claim 1, wherein the breeding farm comprises a breeding net with 80˜100 meshes to prevent invasions from the outside.

3. The method of claim 1, wherein the breeding induction apparatus is formed of flower foam or wood.

4. The method of claim 1, wherein the breeding grooves are formed on either or both sides of the breeding induction apparatus.

5. The method of claim 1, wherein open side of the breeding groove has a geometric shape selected from the group consisting of round shape, elliptical shape, and polygonal shape.

6. The method of claim 1, wherein a diameter of the open side of the breeding groove ranges from 3 mm to 5 mm.

7. The method of claim 1, wherein a depth of the breeding groove ranges from 7 mm to 10 mm.

8. The method of claim 1, wherein a providing density of adult black soldier flies ranges from 8,000 to 12,000 (cm³/head).

9. The method of claim 1, wherein the providing a breeding induction apparatus is performed 3 days after performing the providing adult black soldier flies.

10. The method of claim 1, wherein the induction medium comprises a combination of organic waste and sawdust or rice husk in the ratio of 3:1.

11. The method of claim 1, wherein the induction medium comprises a combination of organic waste and calf starter and sawdust in the ratio of 2:1:1.

12. The method of claim 1, wherein the breeding farm further comprises a stench removing unit to remove a stench emitting from the inside.

13. The method of claim 1 further comprising rearing pupae of black soldier flies in a rearing medium comprising sawdust of which a relative humidity ranges from 0% to 40%.

14. The method of claim 13, wherein the rearing is performed under a condition that a density of pupae ranges from 5,000 to 10,000 (head/240 cm²), temperature is 27±3° C., and the rearing medium comprises a combination of organic waste and sawdust in the ratio of 3:1.

15. The method of claim 10, wherein the organic waste is crushed to be combined.

16. The method of claim 1 further comprising, posterior to the supplying adult black soldier flies, spraying water in the breeding farm two or three times a day to supply water.

17. The method of claim 1, wherein the breeding farm is situated indoors and further comprises an air conditioner to maintain temperature of the breeding farm at 29±5° C.

18. The method of claim 13 further comprising removing a stench emitting from the rearing medium by a stench removing unit.

19. The method of claim 13, wherein the breeding farm or the rearing medium is situated in a vinyl greenhouse having an illuminator or in a building with a transparent ceiling to maintain a photoperiod of the rearing medium at 14L:10D.

20. The method of claim 1, wherein the number of breeding induction apparatuses in the breeding farm of black soldier fly is 8.

21. The method of claim 13, wherein temperature of the rearing medium is maintained at 27±3° C. by an air conditioner.

22. The method of claim 14, wherein the organic waste is crushed to be combined.
